(19) United States
(12) Patent Application Publication
(10) Pub. No.: US 2024/0184138 A1
(43) Pub. Date: Jun. 6, 2024

(54) DIFFRACTIVE LENSES AND RELATED INTRAOCULAR LENSES FOR PRESBYOPIA TREATMENT

(71) Applicant: AMO GRONINGEN B.V., GRONINGEN (NL)

(72) Inventors: Hendrik A. Weeber, Groningen (NL); Robert Rosen, Groningen (NL)

(21) Appl. No.: 18/439,058

(22) Filed: Feb. 12, 2024

Related U.S. Application Data
(62) Division of application No. 17/651,779, filed on Feb. 18, 2022, now Pat. No. 11,914,229.
Application No. 17/651,779 is a continuation of application No. 16/020,928, filed on Jun. 27, 2018, now Pat. No. 11,262,598.
Application No. 16/020,928 is a continuation of application No. 62/525,968, filed on Jun. 28, 2017.
(60) Provisional application No. 62/525,965, filed on Jun. 28, 2017.

(51) Int. Cl. G02C 7/04; A61F 2/14; A61F 2/16; G02C 7/02

(52) U.S. Cl. CPC G02C 7/044; A61F 2/1618; A61F 2/1654; G02C 7/024; G02C 7/042; A61F 2/145; A61F 2240/002; G02C 2202/20

(57) ABSTRACT

Apparatuses, systems and methods for providing improved ophthalmic lenses, particularly intraocular lenses (IOLs), include features for reducing dysphotopsia effects, such as straylight, haloes and glare, in diffractive lenses. Exemplary ophthalmic lenses can include a diffractive profile that distributes light among a near focal length, a far focal length, and one or more intermediate focal length. The diffractive profile provides for minimized or zero step heights between one or more pairs of diffractive zones for reducing visual artifacts.

### CROSS-REFERENCE AND RELATED APPLICATIONS

This application is a divisional of and claims priority to U.S. patent application Ser. No. 17/651,779, filed Feb. 18, 2022, which is a continuation of and claims priority to 16/020,928, filed Jun. 27, 2018, which claims priority to, and the benefit of, under U.S.C. § 119(e) of U.S. Provisional Appl. No. 62/525965, filed on Jun. 28, 2017, all of which are incorporated herein by reference in their entirety.

### BACKGROUND

Embodiments of the present disclosure relate generally to diffractive ophthalmic lenses, and particular embodiments provide methods, devices, and systems for mitigating or treating vision conditions such as presbyopia, often by determining a desired multifocal power profile and selecting a geometry of the diffractive profile that results in a diffractive multifocal lens shape according to the desired power profile and to various parameters of the patient's eye. Embodiments also relate to vision treatment techniques and in particular embodiments, to ophthalmic lenses such as, for example, contact lenses, corneal inlays or onlays, or intraocular lenses (IOLs) including, for example, phakic IOLs and piggyback IOLs (i.e. IOLs implanted in an eye already having an IOL).

Presbyopia is a condition that affects the accommodation properties of the eye. As objects move closer to a young, properly functioning eye, the effects of ciliary muscle contraction and zonular relaxation allow the lens of the eye to change shape, and thus increase its optical power and ability to focus at near distances. This accommodation can allow the eye to focus and refocus between near and far objects.

Presbyopia normally develops as a person ages, and is associated with a natural progressive loss of accommodation. The presbyopic eye often loses the ability to rapidly and easily refocus on objects at varying distances. The effects of presbyopia usually become noticeable after the age of 45 years. By the age of 65 years, the crystalline lens has often lost almost all elastic properties and has only limited ability to change shape.

Along with reductions in accommodation of the eye, age may also induce clouding of the lens due to the formation of a cataract. A cataract may form in the hard central nucleus of the lens, in the softer peripheral cortical portion of the lens, or at the back of the lens. Cataracts can be treated by the replacement of the cloudy natural lens with an artificial lens. An artificial lens replaces the natural lens in the eye, with the artificial lens often being referred to as an intraocular lens or “IOL”.

Multifocal IOLs may, for example, rely on a diffractive optical surface to direct portions of the light energy toward differing focal distances, thereby allowing the patient to clearly see both near and far objects. Multifocal ophthalmic lenses (including contact lenses or the like) have also been proposed for treatment of presbyopia without removal of the natural crystalline lens. Diffractive optical surfaces, either monofocal or multifocal, may also be configured to provide reduced chromatic aberration.

Diffractive monofocal and multifocal lenses can make use of a material having a given refractive index and a surface curvature which provide a refractive power. Diffractive lenses have a diffractive profile which confers the lens with a diffractive power that contributes to the overall optical power of the lens. The diffractive profile is typically characterized by a number of diffractive zones. When used for ophthalmic lenses these diffractive zones are typically annular lens zones, or echelettes, spaced about the optical axis of the lens. Each echelette may be defined by an optical zone, a transition zone between the optical zone and an optical zone of an adjacent echelette, and echelette geometry. The echelette geometry includes an inner and outer diameter and a shape or slope of the optical zone, a height or step height, and a shape of the transition zone. The surface area or diameter of the echelettes largely determines the diffractive power(s) of the lens and the step height of the transition between echelettes largely determines the light distribution between the different powers. Together, these echelettes form a diffractive profile.

A multifocal diffractive profile of the lens may be used to mitigate presbyopia by providing two or more optical powers; for example, one for near vision and one for far vision. The lenses may also take the form of an intraocular lens placed within the capsular bag of the eye, replacing the original lens, or placed in front of the natural crystalline lens. The lenses may be in the form of a contact lens, most commonly a bifocal contact lens, or in any other form mentioned herein.

Multifocal (e.g. diffractive) intraocular lenses (IOLs) are intended to provide a patient with improved vision at different distances, such as near, intermediate and far. The near vision may generally correspond to vision provided when objects are at a distance of equal or less than 1.5 feet from a subject eye. Intermediate vision may generally correspond to vision for objects at a distance between about 1.5 feet and about 5-6 feet from a subject eye. Far vision may generally correspond to vision for objects at any distance greater than about 5-6 feet from a subject eye. Such characterizations of near, intermediate, and far vision correspond to those addressed in

Morlock R, Wirth RJ, Tally SR, Garufis C, Heichel CWD, Patient-Reported Spectacle Independence Questionnaire (PRSIQ): Development and Validation. Am J Ophthalmology 2017; 178:101-114.

Since multifocal IOLs provide multiple focal lengths, the focused image on the retina originating from the focal length that corresponds to the particular viewing distance is overlapping with unfocused images originating from the other focal lengths. This can create visual artifacts for the patient. Also, the transitions between echelettes in a diffractive multifocal may cause glare, halo, or similar visual artifacts; and the severity of said artifacts may increase with an increased number of echelettes. Furthermore, conventional approaches typically provide for near and far vision, but achieve unsatisfactory visual performance at intermediate distances. Relatedly, increasing the number of focal lengths in an IOL can exacerbate the aforementioned visual artifacts. Therefore, multifocal conventional ophthalmic approaches may fail to adequately improve visual performance at intermediate distances.

### BRIEF SUMMARY

Embodiments herein described include IOLs with a first surface and a second surface disposed about an optical axis, and a diffractive profile imposed on one of the first surface or the second surface. The diffractive profile includes a repetitive pattern of at least two echelettes. At least one of the at least two diffractive echelettes in the repetitive pattern is connected to an adjacent echellete by a step height of zero. The zero-step-height transition between at least one adjacent pair of diffractive echelettes is effective to reduce optical aberrations for a user, particularly straylight at the far vision.

Embodiments herein described also include multifocal ophthalmic lenses that have diffractive echelettes directing light to multiple focal lengths in ascending proportions, such that the least light is directed to the near focal length and/or such that the most light is directed to the far focal length. In some cases, at least 50% of the light that passes through the lens can be directed toward the far focal length; and no more than 20% of the light that passes through the lens can be directed toward the near focal length. One or more intermediate focal lengths may be provided.

Embodiments herein described also include ophthalmic lenses that have an optical surface disposed about an optical axis. A diffractive profile is imposed on the optical surface. The diffractive profile includes a set of at least two echelettes, with at least one of the at least two echelettes of the set being connected to an adjacent echelette with a step height of zero, and the set is repeated on the optical surface.

Embodiments herein described also include manufacturing systems for making an ophthalmic lens. Such manufacturing system can include an input that accepts an ophthalmic lens prescription for a patient eye. A module can generate a diffractive profile including a repetitive pattern of at least two echelettes, and at least one of the echelettes in the repetitive pattern is connected to an adjacent echelette by a step height of zero. A manufacturing assembly may fabricate the ophthalmic lens based on the diffractive profile. A manufacturing system may also include an input that accepts an ophthalmic lens prescription for a patient eye. A module can generate a diffractive profile configured to cause a distribution of light among at least three focal lengths including a near focal length, an intermediate focal length, and a far focal length, such that, a first portion of the distribution is directed to the near focal length, a second portion of the distribution is directed to the far focal length, and a third portion of the distribution is directed to the intermediate focal length, the first portion being less than the second portion and less than the third portion. A manufacturing assembly may fabricate the ophthalmic lens based on the diffractive profile.

Embodiments herein described also include methods of designing an intraocular lens. Such methods can include defining a diffractive profile and generating a diffractive lens surface based on the diffractive profile. The diffractive profile can include a repetitive pattern of at least two echelettes, and at least one of the at least two echelletes in the repetitive pattern is connected to an adjacent echelette by a step height of zero. The diffractive profile may also be configured such that a first portion of the distribution is directed to the near focal length, a second portion of the distribution is directed to the far focal length, and a third portion of the distribution is directed to the intermediate focal length, the first portion being less than the second portion and less than the third portion.

### BRIEF DESCRIPTION OF THE DRAWINGS

FIG. 1A illustrates a cross-sectional view of an eye with an implanted multifocal refractive intraocular lens;

FIG. 1B illustrates a cross-sectional view of an eye having an implanted multifocal diffractive intraocular lens;

FIG. 2A illustrates a front view of a diffractive multifocal intraocular lens; FIG. 2B illustrates a cross-sectional view of a diffractive multifocal intraocular lens;

FIG. 3 illustrates a cross-sectional view of an eye having an implanted multifocal diffractive intraocular lens having an intermediate focal length;

FIG. 4 is a graphical representation illustrating aspects of a conventional quadrifocal lens profile;

FIG. 5 is a graphical representation of a generalized multifocal lens profile;

FIG. 6 is a graphical representation illustrating a quadrifocal lens profile according to certain embodiments of this disclosure;

FIG. 7 is a graphical representation of a through-focus point spread function (PSF) according to certain embodiments of this disclosure;

FIG. 8 illustrates a cross-sectional view of a diffractive lens surface having the quadrifocal lens profile of FIG. 6 repeated across the optic;

FIG. 9 is a graphical representation illustrating a quadrifocal lens profile according to

certain embodiments of this disclosure;

FIG. 10 is a graphical representation illustrating a trifocal lens profile according to certain embodiments of this disclosure;

FIG. 11 is a simplified block diagram illustrating a system for generating a diffractive lens surface, in accordance with embodiments;

FIG. 12 illustrates an example process for generating a diffractive lens surface; and

FIG. 13 illustrates an example computing environment for facilitating the systems and processes of FIGS. 11 and 12.

### DETAILED DESCRIPTION

### Contemporary Lens Shapes and Diffractive Profiles

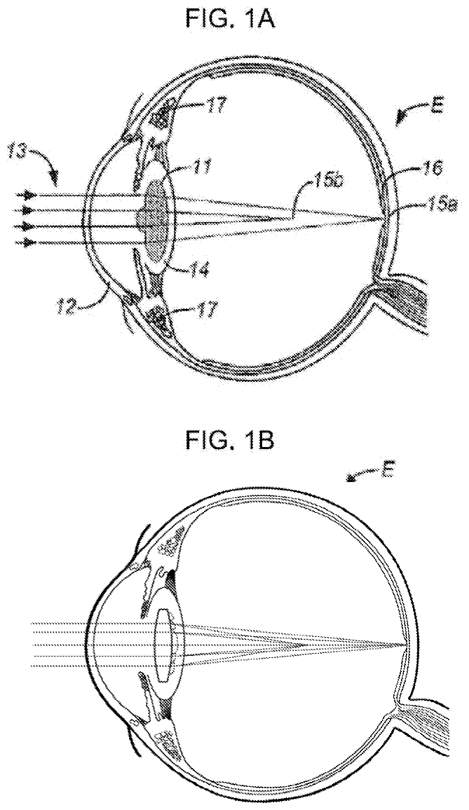

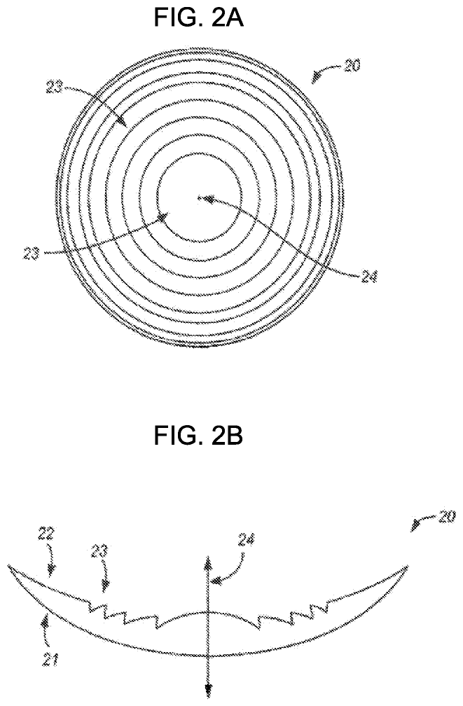

FIGS. 1A, 1B, 2A, and 2B illustrate multifocal IOL lens geometries, aspects of which are described in U.S. Patent Publication No. 2014-0168602 A1, which is hereby incorporated by reference in its entirety.

FIG. 1A is a cross-sectional view of an eye E fit with a multifocal IOL 11. As shown, multifocal IOL 11 may, for example, comprise a bifocal IOL. Multifocal IOL 11 receives light from at least a portion of cornea 12 at the front of eye E and is generally centered about the optical axis of eye E. For ease of reference and clarity, FIGS. 1A and 1B do not disclose the refractive properties of other parts of the eye, such as the corneal surfaces. Only the refractive and/or diffractive properties of the multifocal IOL 11 are illustrated.

Each major face of lens 11, including the anterior (front) surface and posterior (back) surface, generally has a refractive profile, e.g. biconvex, plano-convex, plano-concave, meniscus, etc. The two surfaces together, in relation to the properties of the surrounding aqueous humor, cornea, and other optical components of the overall optical system, define the effects of the lens 11 on the imaging performance by eye E. Conventional, monofocal IOLs have a refractive power based on the refractive index of the material from which the lens is made, and also on the curvature or shape of the front and rear surfaces or faces of the lens. One or more support elements may be configured to secure the lens 11 to a patient's eye.

Multifocal lenses may optionally also make special use of the refractive properties of the lens. Such lenses generally include different powers in different regions of the lens so as to mitigate the effects of presbyopia. For example, as shown in FIG. 1A, a perimeter region of refractive multifocal lens 11 may have a power which is suitable for viewing at far viewing distances. The same refractive multifocal lens 11 may also include an inner region having a higher surface curvature and a generally higher overall power (sometimes referred to as a positive add power) suitable for viewing at near distances.

Rather than relying entirely on the refractive properties of the lens, multifocal diffractive IOLs or contact lenses can also have a diffractive power, as illustrated by the IOL 18 shown in FIG. 1B. The diffractive power can, for example, comprise positive or negative power, and that diffractive power may be a significant (or even the primary) contributor to the overall optical power of the lens. The diffractive power is conferred by a plurality of concentric diffractive zones which form a diffractive profile. The diffractive profile may either be imposed on the anterior face or posterior face or both.

The diffractive profile of a diffractive multifocal lens directs incoming light into a number of diffraction orders. As light enters from the front of the eye, the multifocal lens 18 directs light to form a far field focus 15a on retina for viewing distant objects and a near field focus 15b for viewing objects close to the eye. Depending on the distance from the source of light 13, the focus on retina 16 may be the near field focus 15b instead. Typically, far field focus 15a is associated with Oth diffractive order and near field focus 15b is associated with the 1st diffractive order, although other orders may be used as well.

Bifocal ophthalmic lens 18 typically distributes the majority of light energy into two viewing orders, often with the goal of splitting imaging light energy about evenly (50%:50%), one viewing order corresponding to far vision and one viewing order corresponding to near vision, although typically, some fraction goes to non-viewing orders.

Corrective optics may be provided by phakic IOLs, which can be used to treat patients while leaving the natural lens in place. Phakic IOLs may be angle supported, iris supported, or sulcus supported. The phakic IOL can be placed over the natural crystalline lens or piggy-backed over another IOL. It is also envisioned that the present disclosure may be applied to inlays, onlays, accommodating IOLs, pseudophakic IOLs, other forms of intraocular implants, spectacles, and even laser vision correction.

FIGS. 2A and 2B show aspects of a conventional diffractive multifocal lens 20. Multifocal lens 20 may have certain optical properties that are generally similar to those of multifocal IOLs 11, 18 described above. Multifocal lens 20 has an anterior lens face 21 and a posterior lens face 22 disposed about optical axis 24.

When fitted onto the eye of a subject or patient, the optical axis of lens 20 is generally aligned with the optical axis of eye E. The curvature of lens 20 gives lens 20 an anterior refractive profile and a posterior refractive profile. Although a diffractive profile may also be imposed on either anterior face 21 and posterior face 22 or both, FIG. 2B shows posterior face 22 with a diffractive profile. The diffractive profile is characterized by a plurality of annular diffractive zones or echelettes 23 spaced about optical axis 24. While analytical optics theory generally assumes an infinite number of echelettes, a standard multifocal diffractive IOL typically has at least 9 echelettes, and may have over 30 echelettes. For the sake of clarity, FIG. 2B shows only 4 echelettes. Typically, an IOL is biconvex, or possibly plano-convex, or convex-concave, although an IOL could be plano-plano, or other refractive surface combinations.

Conventional multifocal diffractive lenses typically provide for near and far field vision, neglecting visual performance at intermediate distances. Providing for an additional intermediate focal length by way of additional optical zones, e.g. by providing sets of at least two echelettes, can help to improve the visual performance at intermediate distances. However, as the number of optical zones increases, the risk of visual artifacts also increases. For example, in a quadrifocal diffractive lens having a near focal length, multiple intermediate focal lengths, and a far focal length; visual artifacts such as halos or glare may be visible to a user due to one or more of the boundaries between the optical zones.

### Exemplary Multifocal Lens Shapes:

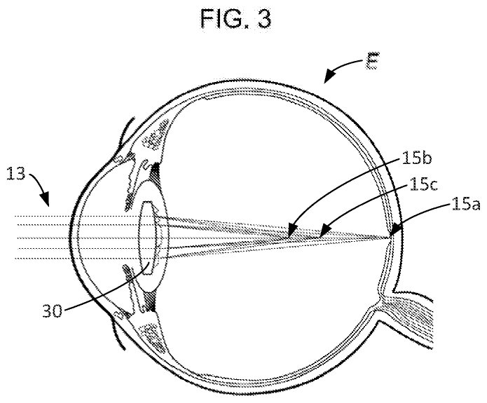

FIG. 3 shows a diffractive multifocal IOL 30 having an intermediate focal length 15c between near and far focal lengths 15b, 15a. The addition of an intermediate focal length 15c can increase the performance of the IOL 30 for users by providing improved visual acuity for viewing objects in the range of about 1.5 feet to about 5-6 feet from the eye. In general, adding a focal length can permit a presbyopic eye to focus more readily on objects at different distances.

The diffractive profile of the diffractive multifocal IOL 30 may provide for the additional focal length beyond the near focal length and far focal lengths described above by employing sets of multiple echelettes. For example, the plurality of concentric diffractive echelettes forming the diffractive profile may be split up into sets of at least two echelettes. The sets are repeating over the optic. The sets of echelettes can direct light 13 toward the near field focus 15b and toward the intermediate field focus 15c. As described above with respect to diffractive multifocal IOLs, the far focus 15a may typically be with a Oth diffractive order, while the near field focus 15b may be associated with a 2^{nd} diffractive order. The intermediate focus 15c may be associated with the 1^{st} diffractive order. However, different configurations are possible. For example, a diffractive multifocal IOL may instead be configured to direct light to the far focal length 15a in the 1^{st} diffractive order, while directing light to the intermediate and near focal lengths 15c and 15b by way of 2^{nd} and 3^{rd} diffractive orders of the echelettes. In other embodiments (a quadrifocal embodiment), an additional intermediate focus (a second intermediate focus) may be provided. Greater or lesser numbers of focuses may be provided as desired in other embodiments.

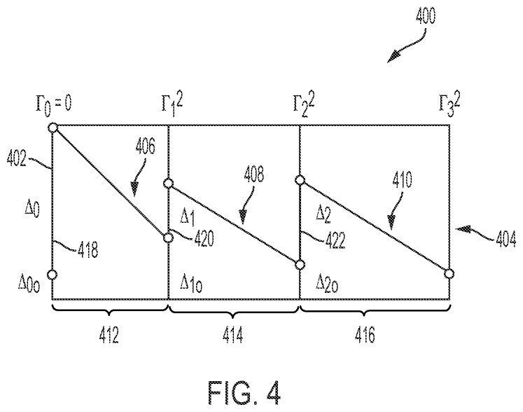

FIG. 4 shows a graphical representation of a portion of a parabolic diffractive profile 400, according to embodiments encompassing a set of 3 echelettes that may repeat. The figure shows the set of 3 echelettes. In the exemplary diffractive profile 400, echelettes 406, 408, and 410 are shown in the X direction (404) from a first, minimum radius ro to a maximum radius r₃². The height of the surface relief pattern (from a plane bisecting the lens) is shown in the Y direction (402) in terms of the phase shift of the echelette (or Δ), and is plotted against the square of the radial distance (r²) from a central axis of the lens. The phase shift corresponds to a physical height or offset of the echelette from an underlying curve of the lens (Δₒ), and may be expressed in terms of wavelength. The echelettes 406, 408, 410 are shown arranged in an A, B, C arrangement, which may be repeated. The diffractive powers of the set of echelettes is driven by the specific geometry of the echelettes, including the change in height Δ₁, Δ₂, over the widths of each echelette 412, 414, 416. The alternating arrangement may be referred to as a saw-tooth profile. Although only three echelettes are shown, it will be understood that any suitable number of echelettes may be positioned on a lens.

Each echelette is connected with each neighboring echelette, where present, by a transition zone. For example, the first echelette 406 connects with the second echelette 408 by a first transition zone 420; and the second echelette 408 connects with the third echelette 410 by a second transition zone 422. The transition zones 420, 422 are step heights 41, 42 from trailing edges of one echelette to leading edges of the next echelette. The first echelette 406 also transitions from a minimum height by third transition zone 418.

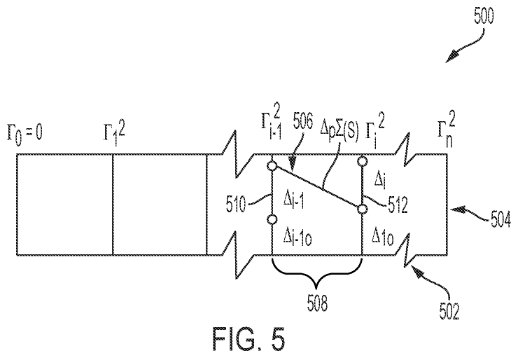

The arrangement of the set of three echelettes 406, 408, 410 in a diffractive profile in FIG. 4 represents a general quadrifocal lens. FIG. 5, however, shows a graphical representation of a generalized set of n echelettes, representing a general profile of a multifocal diffractive lens profile 500 having n add powers, and in total n+1 powers. The profile is shown with the square of the lens radius r² (or ρ) on the X axis 502, and the height of the profile, or phase shift, on the Y axis (504). The diffractive powers of the set of echelettes is driven by the specific geometry of the echelettes, including the radii (r₀, r₁, . . . , r₁, . . . , rₙ).

In a generalized case, where a profile height is maximum at ρᵢ₋₁ and minimum at ρᵢ, the initial maximum profile height 510 may be expressed as a sum of a step height Δᵢ₋₁ and a step offset Δᵢ₋₁ₒ. The step offset is the height offset of the transition zone from the underlying base curve. The following maximum profile height 512 can be expressed as a sum of the following step height Δᵢ and following step offset Δᵢₒ. The slope of profile Δ_{ρi}(ρ) (506) can be expressed in a generalized form as follows.

$$slope = \frac{{Δ}_{io} - ( {Δ}_{i - 1} + {Δ}_{i - 1 o} )}{{ρ}_{i} - {ρ}_{i - 1}}$$

A diffractive profile can provide for multiple focal lengths (or foci) by providing different echelette geometries in series. For example, a diffractive profile having four focal lengths, as described above, can be created by providing three different diffractive echelettes in series (forming a set of three different diffractive echelettes). The three different diffractive echelettes can be repeated, leading to repeated sets of the three different diffractive echelettes, and a diffractive profile over a portion or all of a lens surface. In conventional lenses, the diffractive profile is repeated in a saw-tooth configuration, as shown in FIG. 4.

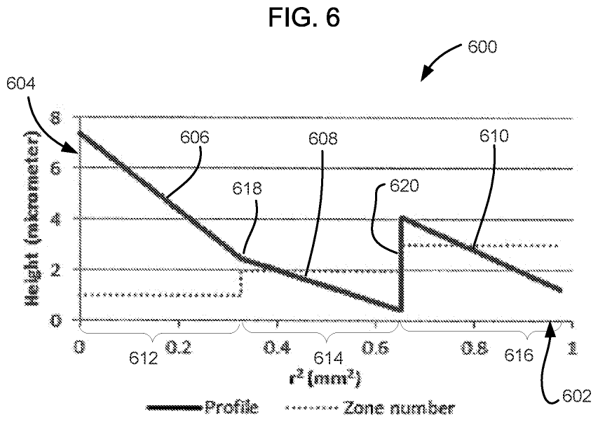

According to certain embodiments of the present disclosure, a diffractive profile can be modified by manipulating the step heights Aj and following step offsets Ajo between echelettes of different echelettes in a set of echelettes. For example, FIG. 6 shows a graphical representation illustrating a modified quadrifocal diffractive lens profile 600 in which a step height between two echelettes has been minimized to be essentially zero. By reducing a step height between two echelettes to zero, or about zero, the potential for that step height to generate visual artifacts such as straylight, rings, or halo can be reduced.

In the diffractive lens profile 600 of FIG. 6, the square of the radius (r² or ρ) is shown on the X axis 602, and the profile height (A) is shown on the Y axis 604. The shape of the diffractive lens profile 600 is represented in relation to the square of the radius (r² or ρ), which is referred to as r-squared space. A first echelette 606 spans a first distance 612; a second echelette 608 spans a second distance 614, and a third echelette 610 spans a third distance 616. Notably, the transition 618 between the first and second echelette 606, 608 has been reduced to a step height of zero by matching an offset of the first echelette 606 with a maximum height of the second echelette 608.

A nonzero step height 620 is still shown between the second and third echelettes 608, 610.

A typical transition zone having a nonzero step height can cause unintended redirection or concentration of light behind the lens, which may contribute to various forms of dysphotopsia. For example, nonzero step height transition zones may cause straylight, halos, glare, or other optical aberrations to appear in the far focal length. As any of the transition zones may cause such optical aberrations, reducing the number of nonzero step-height transition zones can cause a significant reduction in the incidence of such optical aberrations.

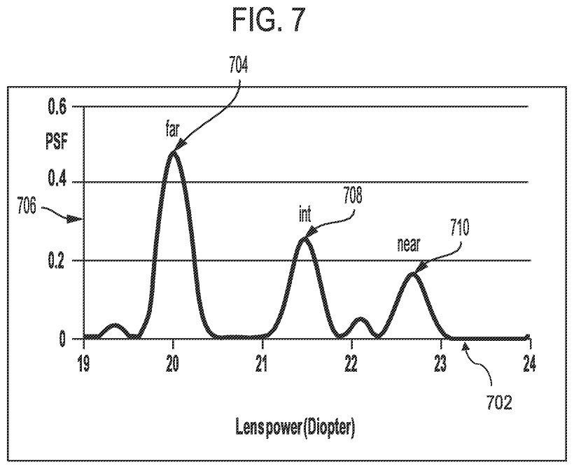

In some embodiments, the reduction in optical aberrations may be enhanced by increasing the amount of light directed toward the far and intermediate focal lengths compared to the amount of light directed toward the near focal length. For example, a diffractive profile may be configured wherein a nonzero percentage of light is diverted to each of a near focal length, an intermediate focal length, and a far focal length, and the amount of light directed to the near focal length can be smaller than the amount directed to any other focal length. According to some embodiments, the echelettes may be arranged to direct light to the far focal length in the 0th diffraction orders, the intermediate in the 1^{st} diffractive order, and the near focal length receives light via the 2nd diffractive order. In other embodiments, the echelettes may be arranged to direct light to the far focal length in the 1^{st} diffractive order, the intermediate focal length in the 2^{nd} diffractive order, and the near focal length receives light by way of the 3^{rd} diffractive order. In some cases, the amount of light directed to the far focal length can be greater than half of the total distribution of light that passes through the lens. The amount of light directed to the near focal length may generally be no more than 20% of the total distribution of light that passes through the lens. A through-focus point spread function (PSF) of such an embodiment is illustrated in FIG. 7. The horizontal axis 702 illustrates the total power of the lens. In this case the lens power for far vision 704 is 20 diopter. The vertical axis 706 illustrates the PSF, or light intensity. The peaks are shown for far vision 704, for intermediate vision 708, and for near vision 710. The peak for near vision 710 is the lower than the peak for intermediate vision 708, and the peak for intermediate vision 708 is lower than the peak for far vision 704. Providing a light distribution, as discussed in regard to FIG. 7, may be provided for an embodiment with a greater or lesser number of focal lengths, which may include a quadrifocal embodiment. For example, in a quadrifocal embodiment, the amount of light directed to the near focal length can be smaller than the amount directed to any other focal length. The amount of light directed to the far focal length can be greater than half of the total distribution of light that passes through the lens. The amount of light directed to the near focal length may generally be no more than 20% of the total distribution of light that passes through the lens. In these embodiments, a diffractive profile having the aforementioned light distribution may or may not include a minimized or zero step height placed between echelettes. In an embodiment with a minimized or zero step height, the minimized or zero step height may be placed between suitable echelettes, particularly between any two echelettes in a repeating set of echelettes.

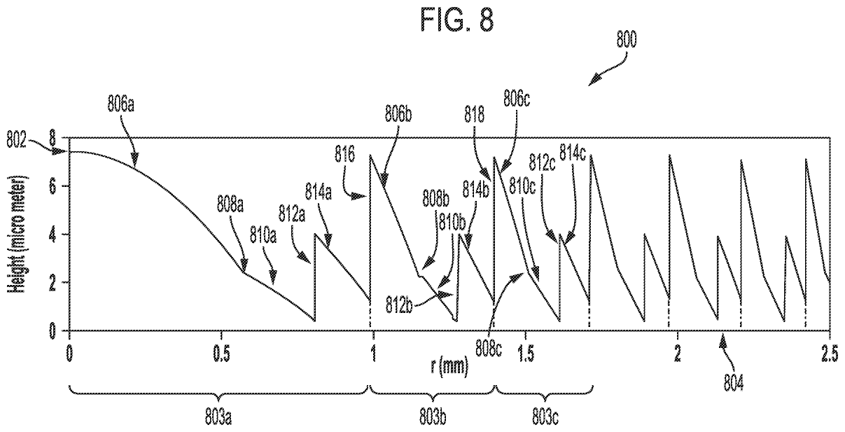

FIG. 8 shows a cross-sectional view of diffractive lens surface 800 having the quadrifocal lens profile that is shown in FIG.6, but here repeated over the optic of the lens.

In the exemplary diffractive lens surface 800, the radius (r) is shown on the X axis 804

and a profile height (Δ) is shown on the Y axis 802.

The diffractive lens surface 800 includes the set 803a of three echelettes 806a, 810a, 814a. The three echelettes 806a, 810a, 814a are the echelettes 606, 608, 610 shown in FIG. 6 (although shown in linear space in FIG. 8, and not in r-squared space as shown in FIG. 6). The set 803a is repeated over the optic to form repeated sets 803b, 803c, and so on, each comprising the same set defined in r²-space, configured to provide different focal lengths at respective diffractive powers. The diffractive profile accordingly includes a repetitive pattern (803a, 803b, 803c) of the echelettes repeated on the optical surface. For example, in first set 803a, a first echelette 806a, second echelette 810a, and third echelette 814a may be provided. The first echelette 806a, second echelette 810a, and third echelette 814a may each have a different profile than each other in r-squared space. The second set 803b may include a first echelette 806b, a second echelette 810b, and a third echelette 814b, each having the same profile in r-squared space as the respective first, second, and third echelettes 806a, 810a, 814a of the first set 803a. The third set 803c may include a first echelette 806c, a second echelette 810c, and a third echelette 814c, each having the same profile in r-squared space as the respective first, second, and third echelettes 806a, 810a, 814a of the first set 803a and the first, second, and third echelettes 806b, 810b, 814b of the second set 803b. The same pattern can repeat for any suitable number of sets.

The echelettes are defined in part by transition zones bounding each respective echelette. For example, regarding the first set 803a, the first echellette 806a is separated from the second echelette 810a by the first transition zone 808a; the second echelette 810a is separated from the third echelette 814a by a second transition zone 812a. The third echelette 814a is separated from the first echelette 806b of the second set 803bby the transition zone 816 between the sets 803a, 803b. Similarly, regarding the second set 803b, the first echellette 806b is separated from the second echelette 810b by the first transition zone 808b; the second echelette 810b is separated from the third echelette 814b by a second transition zone 812b. The third echelette 814b is separated from the first echelette 806c of the third set 803c by the transition zone 818 between the sets 803b, 803c. Regarding the third set 803c, the first echellette 806c is separated from the second echelette 810c by the first transition zone 808c; the second echelette 810c is separated from the third echelette 814c by a second transition zone 812c. The pattern repeats across the additional sets of echelettes.

As with conventional diffractive lenses, some of the transition zones (e.g. zones 812a, 816, 812b, 818) may have a nonzero step height. However, in accordance with embodiments, at least one pair of echelettes (e.g. zones 806a, 810a) is separated by a transition zone 808a having a step height of zero. At least one of the echelettes is connected to an adjacent echelette by a step height of zero. As the echelettes repeat across sets, further adjacent echelettes (e.g. echelettes 806b and 810b; 806c and 810c) may be separated by transition zones having step heights of zero (e.g. transition zones 808b, 808c).

Although the exact number of repeating sets shown in FIG. 8 is about six, any suitable number of repeating sets may be applied to a lens depending on the specific geometry of the echelettes and the width of the lens. For example, in certain embodiments, at least two sets repeating radially outward may be utilized. In some cases, the profile can extend over a total radius of approximately 2.5 millimeters (mm), as shown; but in other cases, the profile may extend from as little as about 1 mm to as much as about 4 mm.

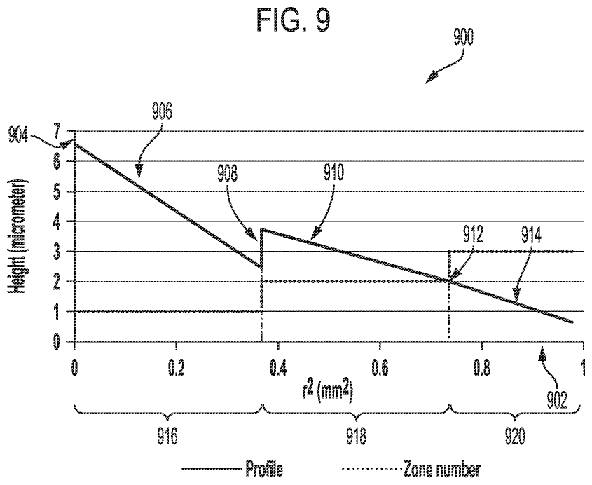

FIG. 9 shows a graphical representation illustrating a second quadrifocal lens profile 900 according to certain embodiments of this disclosure. The quadrifocal lens profile 900 is shown in terms of profile height (or Δ), or phase shift, on the Y axis 904 against the square of the radius (or p) on the X axis 902 (in r-squared space). The profile 900 defines a set of three distinct echelettes 906, 910, 914 each spanning a respective portion 916, 918, 920 of the lens. In the quadrifocal lens profile 900, for an A, B, C arrangement of three distinct echelettes, the minimum or zero step height 912 is positioned at the B-C transition between the second echelette 910 and the third echellete 914. In this example, the minimum or zero step height 912 is convex, as the preceding or second echelette 910 is less steep than the subsequent or third echelette 914. A non-zero step height 908 connects the first echelette 906 to the second echelette 910.

As discussed above, the positioning of the minimized or zero step height may be adjusted. The example in FIGS. 6 and 8 shows a configuration wherein, for an A, B, C arrangement of three distinct echelettes, the minimum or zero step height is positioned at the A-B transition. The example in FIG. 9 shows a configuration wherein, for an A, B, C arrangement of three distinct diffractive zones, the minimum or zero step height is positioned at the B-C transition. The transition having minimum or zero step height is convex, as an echelette 910 merged at its respective minimum height with a steeper echelette 914. In FIG. 6, the transition having minimum or zero step height is concave, as a steeper echelette 606 merged at its respective minimum height with a less steep echelette 608.

A concave or convex transition may influence the performance of the profile, and the manufacturability. The size or extent of concave transitions may be minimized if lens is manufactured by molding. In contrast, the size or extent of convex transitions may be minimized if the lens is manufactured by lathe cutting.

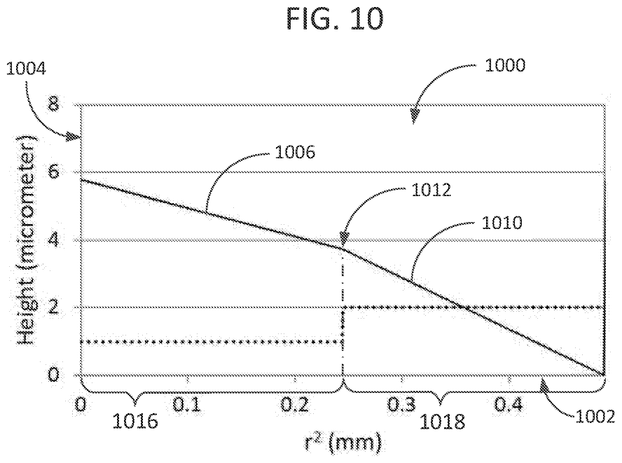

FIG. 10 shows a graphical representation illustrating a trifocal lens profile 1000 according to certain embodiments of this disclosure. The trifocal lens profile 1000 is shown in terms of profile height (or 4), or phase shift, on the Y axis 1004 against the square of the radius (or p) on the X axis 1002 (in r-squared space). The profile 1000 defines a set of two distinct echelettes 1006, 1010 each spanning a respective portion 1016, 1018 of the lens. In the trifocal lens profile 1000, for an A, B arrangement of two distinct echelettes, the minimum or zero step height 1012 is positioned at the A-B transition between the first echelette 1006 and the second echelette 1010. In this example, the minimum or zero step height 1012 is convex, as the preceding or first echelette 1006 is less steep than the subsequent or second echelette 1010. The set of echelettes comprising the first echelette 1006 and second echelette 1010 may be repeated over the optic of the lens for any number of repetitions, as desired.

Any of the embodiments of lens profiles discussed herein may be apodized to produce a desired result. The apodization may result in the step heights and step offsets of the repeated sets being varied according to the apodization. The sets, however, are still considered to be repeating sets over the optic of the lens.

The structures and methods discussed herein may be used to produce a lens having any number of focal lengths (monofocal, bifocal, trifocal, quadrifocal, etc.), and the diffractive profiles discussed herein may be used to produce any number of focal points (at least one focal point). The diffractive profiles may be applied to cover an annulus of the first surface or the second surface.

The lens may be characterized as a monofocal lens or extended depth of focus lens.

### Systems and Methods for Determining Lens Shape:

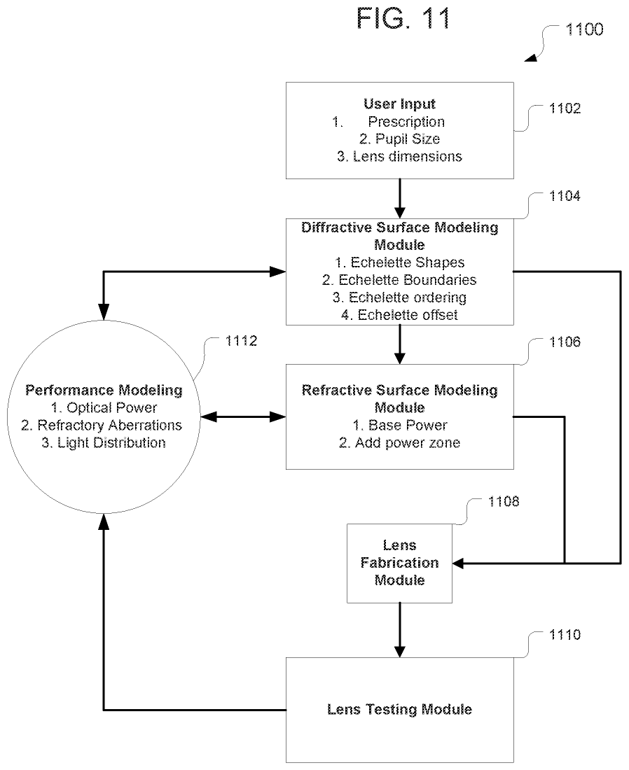

FIG. 11 is a simplified block diagram illustrating a system 1100 for generating an ophthalmic lens based on a user input.

The system 1100 includes a user input module 1102 configured to receive user input defining aspects of the user of a lens and of the lens itself. Aspects of a lens may include anatomical dimensions like pupil size performance, and lens dimensions, among other attributes, and a diffractive lens prescription, which may be a multifocal prescription. A lens prescription can include, for example, a preferred optical power or optical power profile for correcting far vision and an optical power or optical power profile for near vision. In some cases, a lens prescription can further include an optical power or optical power profile for correcting intermediate vision at two, or in some cases more than two intermediate foci, which may fall between the optical powers or ranges of optical powers described above. A pupil size performance can include a pupil radius of a patient and the visual field to be optimized. These parameters can also be related to patient's life style or profession, so that the design incorporates patient's visual needs as a function of the pupil size. Lens dimensions can include a preferred radius of the total lens, and may further include preferred thickness, or a preferred curvature of one or the other of the anterior surface and posterior surface of the lens.

A diffractive surface modeling module 1104 can receive information about the desired lens from the user input module 1102, and can determine aspects of a multizonal lens. For example, the modeling module 1104 can determine the shape of one or more echelettes of the diffractive profile of a diffractive lens, including the positioning, width, step height, and curvature needed to fulfill the prescription for each set of the echelettes, as well as the positioning of each set of echelettes. The multizonal diffractive surface modeling module 1104 can further determine the shapes of transition steps between echelettes. For example, transition steps may be smoothed or rounded to help mitigate optical aberrations caused by light passing through an abrupt transition. Such transition zone smoothing, which may be referred to as a low scatter profile, can provide for reductions in dysphotopsia by reducing the errant concentration of incident light behind the lens by the transition zones. By way of further example, echelette ordering, echelette offsets, and echelette boundaries may be adjusted to adjust the step heights between some adjacent echelettes. In particular, the multizonal diffractive surface modeling module can determine echelette offsets to set one or more step heights at echelette transitions to zero, or approximately zero, by these or similar methods.

The diffractive surface modeling module 1104 can be configured to generate performance criteria 1112, e.g. via modeling optical properties in a virtual environment. Performance criteria can include the match of the optical power profile of the multizonal lens with the desired optical power profile based on the lens prescription. The performance criteria can also include the severity of diffractive aberrations caused by lens surface. In some cases, the multizonal surface modeling module 1104 can provide a lens surface to a lens fabrication module for facilitating the production of a physical lens, which can be tested via a lens testing module 1110 for empirically determining the performance criteria 1112, so as to identify optical aberrations and imperfections not readily discerned via virtual modeling, and to permit iteration.

A refractive surface modeling module 1106 can receive information from the user input 1102 and multifocal surface modeling modules 1104 in order to determine refractive aspects of the lens. For example, provided with a multifocal prescription and a set of diffractive powers that can be generated by a diffractive profile, the refractive surface modeling module 1106 can provide a refractive geometry configured to provide a base power which, when combined with the diffractive surface, meets the requirements of the lens prescription. The refractive surface modeling module 1106 can also generate performance criteria 1112, and can contribute to providing a lens surface to a lens fabrication module 1108 for facilitating the production of the physical lens.

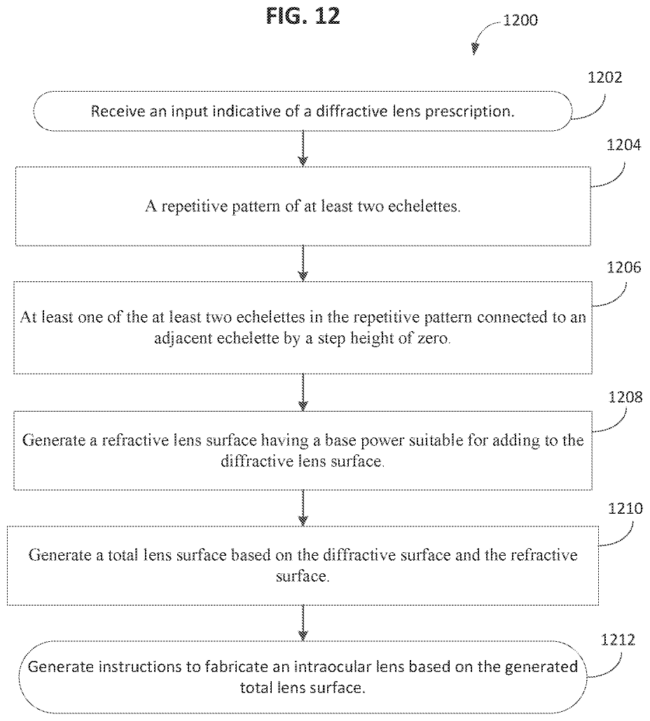

FIG. 12 is an example process 1200 for generating a diffractive lens surface, in accordance with embodiments. The process 1200 may be implemented in conjunction with, for example, the system 1100 shown in FIG. 11. Some or all of the process 1200 (or any other processes described herein, or variations, and/or combinations thereof) may be performed under the control of one or more computer systems configured with executable instructions and may be implemented as code (e.g., executable instructions, one or more computer programs, or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

The process 1200 includes receiving an input indicative of a diffractive lens prescription (act 1202). The input can include, e.g., a desired optical power profile for correcting impaired distance vision, a desired optical power profile for correcting impaired intermediate distance vision, a desired optical power profile for accommodating near vision, and any suitable combination of the above. Based on a desired optical power profile, a diffractive profile can be generated including a repetitive pattern of at least two echelettes (act 1204). At least one of the at least two echelettes in the repetitive pattern may be connected to an adjacent echelette by a step height of zero (act 1206).

The diffractive lens profile of the multizonal diffractive lens surface may be used in combination with a known refractive base power. To that end, a refractive lens surface may be generated having a base power that, in combination with the diffractive lens surface, meets the diffractive lens prescription (act 1208). A total lens surface can be generated based on both the refractive lens surface and the diffractive lens surface (act 1210). The refractive lens surface can include a refractive lens curvature on the anterior surface of the lens, the posterior surface of the lens, or both. Instructions can be generated to fabricate an intraocular lens based on the generated total lens surface (act 1212).

### Computational Methods:

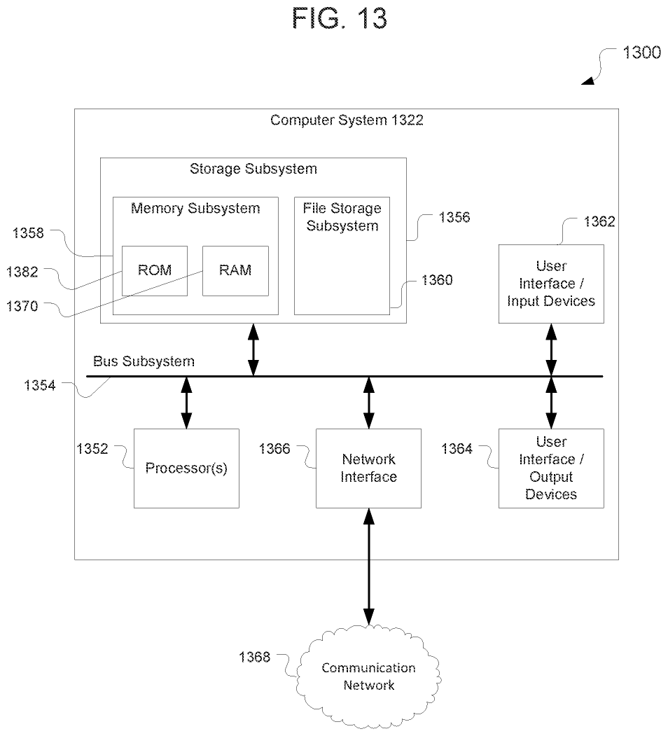

FIG. 13 is a simplified block diagram of an exemplary computing environment 1300 that may be used by systems for generating the continuous progressive lens surfaces of the present disclosure. Computer system 1322 typically includes at least one processor 1352 which may communicate with a number of peripheral devices via a bus subsystem 1354. These peripheral devices may include a storage subsystem 1356 comprising a memory subsystem 1358 and a file storage subsystem 1360, user interface input devices 1362, user interface output devices 1364, and a network interface subsystem 1366. Network interface subsystem 1366 provides an interface to outside networks 1368 and/or other devices, such as the lens fabrication module 1108 or lens testing module 1110 of FIG. 11.

User interface input devices 1362 may include a keyboard, pointing devices such as a mouse, trackball, touch pad, or graphics tablet, a scanner, foot pedals, a joystick, a touchscreen incorporated into the display, audio input devices such as voice recognition systems, microphones, and other types of input devices. User input devices 1362 will often be used to download a computer executable code from a tangible storage media embodying any of the methods of the present disclosure. In general, use of the term “input device” is intended to include a variety of conventional and proprietary devices and ways to input information into computer system 1322.

User interface output devices 1364 may include a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from computer system 1322 to a user.

Storage subsystem 1356 can store the basic programming and data constructs that provide the functionality of the various embodiments of the present disclosure. For example, a database and modules implementing the functionality of the methods of the present disclosure, as described herein, may be stored in storage subsystem 1356. These software modules are generally executed by processor 1352. In a distributed environment, the software modules may be stored on a plurality of computer systems and executed by processors of the plurality of computer systems. Storage subsystem 1356 typically comprises memory subsystem 1358 and file storage subsystem 1360. Memory subsystem 1358 typically includes a number of memories including a main random access memory (RAM) 1370 for storage of instructions and data during program execution.

Various computational methods discussed above, e.g. with respect to generating a multizonal lens surface, may be performed in conjunction with or using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like.

What is claimed is:

1. An ophthalmic lens, comprising:
a first surface and a second surface disposed about an optical axis; and
a diffractive profile imposed on one of the first surface or the second surface, wherein:
the diffractive profile includes a repetitive pattern of at least two echelettes; and
at least one of the at least two echelettes in the repetitive pattern is connected to an adjacent echelette by a step height of zero.

2. The lens of claim 1, wherein the repetitive pattern is of three echelettes.

3. The lens of claim 2, wherein the lens is a quadrifocal lens.

4. The lens of claim 1, wherein the lens is a trifocal lens.

5. The lens of claim 1, wherein the lens is an extended depth of focus lens.

6. The lens of claim 1, wherein the lens is a monofocal lens.

7. The lens of claim 1, wherein the diffractive profile covers an area of the first surface or the second surface.

8. The lens of claim 1, wherein the diffractive profile covers an annulus of the first surface or the second surface.

9. The lens of claim 1, wherein the diffractive profile creates at least one focal point.

10. The lens of claim 9, wherein the diffractive profile creates at least two focal points.

11. The lens of claim 1, wherein the diffractive profile is operable to reduce optical aberration at the far focal length.

12. The lens of claim 1, wherein the repetitive pattern includes a form of apodization.

13. An ophthalmic lens, comprising:
a first surface and a second surface disposed about an optical axis; and
a diffractive profile imposed on one of the first surface or the second surface, and configured to cause a distribution of light among at least three focal lengths including a near focal length, an intermediate focal length, and a far focal length, such that:
a first portion of the distribution is directed to the near focal length,
a second portion of the distribution is directed to the far focal length, and
a third portion of the distribution is directed to the intermediate focal length, the first portion being less than the second portion and less than the third portion.

14. The lens of claim 13, wherein the first portion of the distribution is smaller than a respective portion of the distribution directed to any other focal length of the at least three focal lengths.

15. The lens of claim 13, wherein the first portion of the distribution of light is no more than 20% of the distribution of light.

16. The lens of claim 13, wherein the second portion of the distribution of light is at least 50% of the distribution of light.

17. An ophthalmic lens, comprising:
an optical surface disposed about an optical axis; and
a diffractive profile imposed on the optical surface, wherein:
the diffractive profile includes a set of at least two echelettes, with at least one of the at least two echelettes of the set being connected to an adjacent echelette with a step height of zero, and
the set is repeated on the optical surface.

18. The ophthalmic lens of claim 17, wherein two of the at least two echelettes of the set have a different profile than each other in r-squared space.

19. The ophthalmic lens of claim 17, wherein the at least two echelettes of the set comprise a first echelette and a second echelette, and the step height of zero is between the first echelette and the second echelette.

20. The ophthalmic lens of claim 17, wherein the lens is a trifocal lens.

21. The ophthalmic lens of claim 17, wherein the at least two echelettes of the set comprise a first echelette, a second echelette, and a third echelette, with the second echelette positioned between the first echelette and the third echelette, and the third echelette being positioned radially outward of the second echelette, and the step height of zero is between the first echelette and the second echelette.

22. The ophthalmic lens of claim 17, wherein the at least two echelettes of the set comprise a first echelette, a second echelette, and a third echelette, with the second echelette positioned between the first echelette and the third echelette, and the third echelette being positioned radially outward of the second echelette, and the step height of zero is between the second echelette and the third echelette.

23. The ophthalmic lens of claim 17, wherein the lens is a quadrifocal lens.

24. The ophthalmic lens of claim 17, wherein the set is repeated on the optical surface radially outward from the optical axis to form at least two of the sets on the optical surface.

25. The ophthalmic lens of claim 17, wherein the set is repeated on the optical surface radially outward from the optical axis to form at least six of the sets on the optical surface.

26. The ophthalmic lens of claim 17, wherein the set is repeated on the optical surface to form a repeated set, the repeated set being apodized.

27. The lens of claim 17, wherein the lens is an extended depth of focus lens.

28. The lens of claim 17, wherein the lens is a monofocal lens.

29. A manufacturing system for making an ophthalmic lens, the system comprising:
an input that accepts an ophthalmic lens prescription for a patient eye;
a first module configured to generate a diffractive profile based on the ophthalmic lens prescription, wherein:
the diffractive profile includes a repetitive pattern of at least two echelettes, and
at least one of the at least two echelettes in the repetitive pattern is connected to an adjacent echelette by a step height of zero; and
a manufacturing assembly that fabricates the ophthalmic lens based on the diffractive profile.

30. A method of designing an intraocular lens, the method comprising:
defining a diffractive profile including:
a repetitive pattern of at least two echelettes, and
at least one of the at least two echelettes in the repetitive pattern is connected to an adjacent echelette by a step height of zero; and
generating a diffractive lens surface based on the diffractive profile.

31. A manufacturing system for making an ophthalmic lens, the system comprising:
an input that accepts an ophthalmic lens prescription for a patient eye;
a first module configured to generate a diffractive profile based on the ophthalmic lens prescription, wherein:
the diffractive profile is configured to cause a distribution of light among at least three focal lengths including a near focal length, an intermediate focal length, and a far focal length, such that:
a first portion of the distribution is directed to the near focal length,
a second portion of the distribution is directed to the far focal length, and
a third portion of the distribution is directed to the intermediate focal length, the first portion being less than the second portion and less than the third portion; and
a manufacturing assembly that fabricates the ophthalmic lens based on the diffractive profile.

32. A method of designing an intraocular lens, the method comprising:
defining a diffractive profile configured to cause a distribution of light among at least three focal lengths including a near focal length, an intermediate focal length, and a far focal length, such that:
a first portion of the distribution is directed to the near focal length,
a second portion of the distribution is directed to the far focal length, and
a third portion of the distribution is directed to the intermediate focal length, the first portion being less than the second portion and less than the third portion; and
generating a diffractive lens surface based on the diffractive profile.
